(19) United States
(12) Patent Application Publication
(10) Pub. No.: US 2025/0291953 A1
(43) Pub. Date: Sep. 18, 2025

(54) SYSTEM AND METHOD FOR PROTECTING PATIENTS THERAPEUTIC TREATMENT CONTEXT USED FOR TRAINING ARTIFICIAL INTELLIGENCE SYSTEMS

(71) Applicant: Lifeguard Health Networks, Inc., Radnor, PA (US)

(72) Inventors: Stephen Malaby, Devon, PA (US); Theodore Goldstein, Los Altos, CA (US)

(21) Appl. No.: 19/068,782

(22) Filed: Mar. 3, 2025

Related U.S. Application Data
(60) Provisional application No. 63/565,656, filed on Mar. 15, 2024.

(51) Int. Cl. G06F 21/62; G16H 10/60

(52) U.S. Cl. CPC G06F 21/6254; G16H 10/60

(57) ABSTRACT

A method disclosed herein is employed in a computerized system. Initially, categories are defined in the memory, encompassing both identifier categories for direct identifiers of personally identifiable information and enumerated categories for objects related to such information but distinct from direct identifiers. Medical facts for numerous patients are acquired and processed into fact vectors based on these categories using processors within the system. A de-identification process is conducted to remove direct identifiers from the fact vectors, and a fictionalization process is carried out to alter enumerated objects. The resultant de-identified and fictionalized medical facts are stored in memory as a training dataset. Utilizing this dataset, the system trains an untrained artificial intelligence (AI) model, transforming it into a trained AI model through processor-based training procedures.

### CROSS-REFERENCE TO RELATED APPLICATIONS

This application claims the benefit of U.S. Provisional Appl. No. 63/565,656 filed Mar. 15, 2024, which is incorporated herein by reference in its entirety. This application is also filed concurrently with U.S. Non-Provisional Appl. No.__/______and entitled “System and Method for Therapeutic Event Management and Value Attribution (Proof of Value) In Decentralized Health Information Network” (Atty. Dkt. No. 266-0006US) and U.S. Non-Provisional Appl. No.__/______and entitled “System and Method for AI Assisted Therapeutic Micro-Interventions in Care at Home” (Atty. Dkt. No. 266-0007US), both of which are incorporated herein by reference in their entireties.

### FIELD OF THE DISCLOSURE

The subject matter of the present disclosure is directed to techniques for protecting the therapeutic context of a patient's protected health information in medical data used for training an Artificial Intelligence System.

### BACKGROUND OF THE DISCLOSURE

Medical information is regulated by privacy rules in the Health Insurance Portability and Accountability Action of 1996 (HIPPA) in the United States. Similar regulations apply in other countries around the world. This medical information, referred to as Protected Health Information (PHI), cannot be safely used for AI training without being de-identified to protect the identity of the patient. De-identification is a common practice in wide use today.

Under the HIPAA privacy rules, for instance, Protected Health Information is defined as any information that relates to the past, present, or future physical or mental health or condition of an individual; the provision of health care to an individual; or the past, present, or future payment for the provision of health care to an individual. Essentially, the protected Health Information includes any information that could identify an individual or could be used with other information in a record set to identify an individual. Identifiers in a medical record include any information that can be used to identify an individual in a medical record. The Department of Health and Human Services (HHS) lists the 18 HIPAA identifiers.

The HIPAA privacy rules aim to safeguard personal health information, allowing only certain authorized uses and disclosures. However, recognizing the potential benefits of utilizing large datasets for health studies, the rules permit the creation of non-identifiable health information through de-identification standards. There are two methods for de-identification: one involves a formal determination by a qualified expert, while the other entails removing specified identifiers so that the remaining information cannot identify individuals alone or in combination with other data.

Despite these precautions, there is still a risk that de-identified data could potentially be linked back to individuals. Therefore, the HIPAA guidelines emphasize the importance of carefully applying these methods to minimize such risks. Detailed guidance on de-identification methods can be found in the “Guidance Regarding Methods for De-identification of Protected Health Information in Accordance with the Health Insurance Portability and Accountability Act (HIPAA) Privacy Rule” available at Methods for De-identification of PHI|HHS.gov.

At present, AI technology is vulnerable to hacking techniques that can extract sensitive information from models. In some instances, for example, hacking methods may still be able to extract information from AI models trained with de-identified information, using manipulation techniques that triangulate established or speculated facts to reveal a target fact from within the AI model. For example, the human immunodeficiency virus (HIV) status of a well-known political figure could be deduced from an AI model by triangulating publicly available facts to narrow the potential health information. Knowing this figure was treated for different conditions at specific hospital(s) on a certain day, the potential outputs from an AI model can be narrowed to the particular person with higher probability, and the HIV status question can be posed with these narrowing constraints.

The subject of the present disclosure is directed to better protect the identity of individuals and undermine the triangulation techniques just described.

### SUMMARY OF THE DISCLOSURE

As disclosed herein, a method is used in a computerized system. Fact categories are defined in memory of the computerized system. The fact categories include one or more identifier categories and include one or more enumerated categories. The one or more identifier categories categorizes direct identifiers of personally identifiable information. The one or more enumerated categories categorize enumerated objects that are separate from the direct identifiers. The enumerated objects are at least related to information that is personally identifiable.

Medical facts for a plurality of patients are obtained with one or more processors of the computerized system, and the medical facts are vectorized, with the one or more processors, into fact vectors according to the fact categories. In a de-identification process with the one or more processors, any given one or more of the direct identifiers for the fact vectors in the one or more identifier categories are de-identified. In a fictionalization process with the one or more processors, any given one or more of the enumerated objects for the fact vectors in the one or more enumerated categories are fictionalized.

The medical facts resulting from the de-identification process and the fictionalization process are stored in the memory as a training dataset. An untrained artificial intelligence (AI) model of the computer system is trained, with the one or more processors, into a trained AI model using the training dataset. The trained AI model is tested, with the one or more processors, for functional utility and resistance to triangulation attacks.

As disclosed herein, a computerized system comprises a memory, an interface, and one or more processors. The memory stores an untrained artificial intelligence (AI) model and stores fact categories. The fact categories include one or more identifier categories and include one or more enumerated categories. The one or more identifier categories categorize direct identifiers of personally identifiable information. Meanwhile, the one or more enumerated categories categorize enumerated objects that are separate from the direct identifiers. The enumerated objects are at least related to information that is personally identifiable. The interface is configured to obtain medical facts for a plurality of patients.

The one or more processors are in operable communication with the memory and the interface. The one or more processors are configured to vectorize the medical facts into fact vectors according to the fact categories. The one or more processors are configured to de-identify in a de-identification process any given one or more of the direct identifiers for the fact vectors in the one or more identifier categories, and to fictionalize in a fictionalization process any given one or more of the enumerated objects for the fact vectors in the one or more enumerated categories. The one or more processors are configured to store the medical facts resulting from the de-identification process and the fictionalization process in the memory as a training dataset. The one or more processors are configured to train, using the training dataset, the untrained AI model into a trained AI model for storage in the memory, and to test the trained AI model for functional utility and resistance to triangulation attacks.

The foregoing summary is not intended to summarize each potential configuration or every aspect of the present disclosure.

### BRIEF DESCRIPTION OF DRAWINGS

FIG. 1 illustrates an example of a health information network according to the present disclosure.

FIG. 2 illustrates a process of altering patient health information in medical data used for training artificial intelligence (AI) systems.

FIG. 3 illustrates a process according to the present disclosure in additional detail.

FIG. 4A illustrates a diagram of altering medical facts found in medical data to be used in training AI systems.

FIG. 4B illustrates a diagram of altering clusters of medical facts to protect protected health information found in medical data to be used in training AI systems.

FIG. 5 illustrates a natural language processing platform for creating training data to be used in training an AI model in a computing environment.

FIG. 6 illustrates a process for training and deploying a primary deep neural network to process an input dataset to produce results according to a primary purpose of the present disclosure.

FIG. 7 illustrates a process for training and deploying a preparatory deep neural network for processing an underlying dataset to produce a training data for use by the primary deep neural network.

### DETAILED DESCRIPTION OF THE DISCLOSURE

U.S. Publication No. US2014/0207486 to Carty et al and assigned to Lifeguard Health Networks, Inc. is hereby incorporated by reference in its entirety as background information and to aid in the understanding of more advanced concepts discussed herein.

Systems and methods described herein disclose a network enabled (e.g., TCIP/IP enabled) continuity of care network, including one or more computing devices, sensors, and systems for communications and data sharing. These networks enable people on a caregiver team to receive and send medically relevant communications with a patient or a patient representative in response to health events, incidents, questions, observations, trends, and the like. A continuity of care network may be doctor-initiated and payer or insurer approved, and the continuity of care network is part of one possible network arrangement of this disclosure. In many arrangements, patients are invited onto the system by a healthcare provider or a medical-related entity with a relationship to the patient's care. Because healthcare often involves a support team around the patient, arrangements of the disclosed network system allow patients to invite family, friends, and other health advocates to join the system as a patient representative. One or more computing devices for each system participant may be joined into the network/system to run or access software related to the techniques herein and access associated functionality.

The disclosed system proposes technology and solutions through a collaborative care network, including devices having software made available for use by various participants in the healthcare ecosystem and marketplace. For example, depending upon the arrangement, software delivering all or part of the solution may reside on mobile devices, computers, thin clients, or intelligent devices of any kind that are commonly used by patients, patient representatives, healthcare professionals, insurers, government, medically related entities such as institutions, health systems, research entities, and all manner of healthcare provider organizations. As implemented, the software may have multiple widespread application areas. Healthcare providers and other healthcare related entities (such as insurers and institutes) may use a version of software (e.g., application) adapted for endpoint operating systems such as Microsoft Windows or Mac OS, or server operating systems (e.g., for SaaS solutions) such as Linux or Windows Server. In addition, some embodiments allow providers and other entities access to software functionality through (i) mobile applications (e.g., IOS, Android, Java); (ii) a web portal accessed through a web browser on any device; or (iii) any information access and/or delivery system such as virtual or augmented reality tools. In some embodiments, these options may be facilitated through known client-server protocols using computer servers in a data center, public cloud, or other known computing resources.

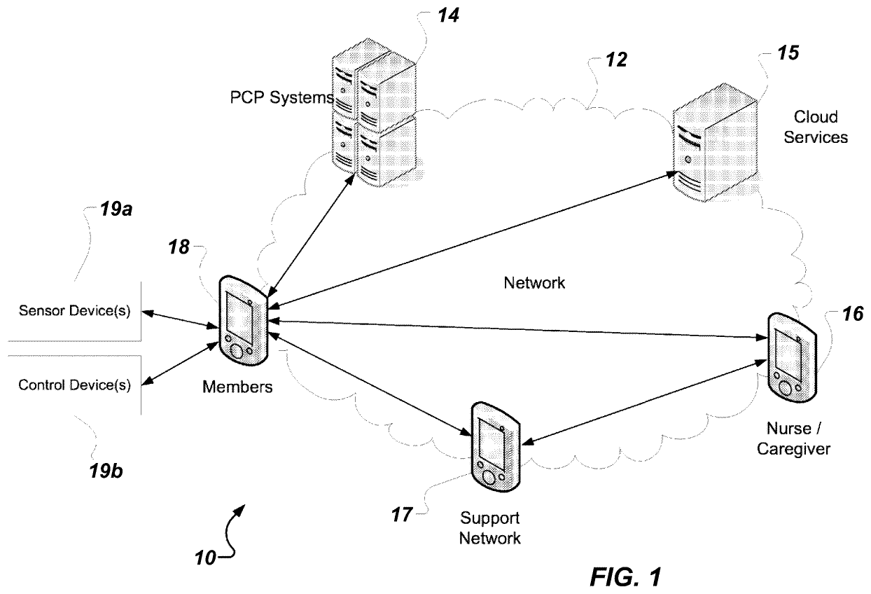

FIG. 1 shows an exemplary continuity of care network system 10. As shown, the care network system 10 is decentralized with computing power, data capture and monitoring and other functionality distributed and available at each of the various components. Patients or their representatives typically interface with/connect to the care network system 10 with patient devices 18. Patient device 18 may be an edge-of-network computing device (e.g., a computer or mobile device such as a smartphone or tablet), optionally configured to monitor patient data based upon a specified health service policy. For example, a device may monitor the patient's biotelemetry, location, or other items depending upon the capability of the device. The patient's device (e.g., client device) 18 may also receive inputs from other devices such as sensors 19a or control devices 19b. Patient device 18 may transmit patient data across one or more networks 12 to other devices and systems in the care network system 10, such as one or more representative devices (e.g., client device) 17, a nurse or other caregiver device (e.g., client device) 16, one or more primary care provider systems 14, and servers/databases such as cloud services systems 15. Of course, the care network system 10 may include any number of additional devices and systems or may omit one or more devices or systems shown in FIG. 1.

Patient representative devices (e.g., client device) 17 may be used by those appointed by or on behalf of the patient, such as trusted family, friends, and other caregivers of a patient to assist with daily therapy compliance, program adherence, communications, health management, and the like. The care network system 10 may include multiple patient representative devices 17 coupled to (i.e., in communication with) a patient device 18, thereby providing a few-to-one or many-to-one support network for the patient. By including multiple people's devices with customized privacy and intervention alert levels within the network, the responsibility of monitoring patient data may be distributed amongst a patient's family or other care representatives or network participants. Additionally, such a group may provide monitoring redundancy, for example if one representative does not have access (e.g., internet access) others in the patient's support network may monitor patient's data. In addition, aspects of the care network system 10 related to the patient may be dynamic, expanding and contracting in response to a patient's needs, schedule, and the like.

Representative devices 17 and nurse/caregiver device 16 may be any type of computing devices, such as any computing endpoint or mobile computing device, thereby enabling monitoring the patient substantially all times. Devices on the care network system 10 may receive substantially real-time health data from patient device 18 to allow for a timely and effective intervention mechanisms.

The care network system 10 may also include one or more healthcare provider systems 14 (e.g., PCP—or primary care provider systems). These provider systems 14 may periodically receive patient data from patient device 18, optionally as relayed through one or more servers. Medical practitioners may use provider systems 14 to diagnose patients, monitor effectiveness of therapy regimens, access EHR systems and facilitate similar functions or other tasks related to the provision of healthcare. Care network system 10 may allow medical providers to monitor and diagnose many patients while simultaneously allowing designated family, friends, nurses, and the like to also monitor the patient's therapy regimen compliance, potential emergencies and other situations or statuses.

The care network system 10 may include or access cloud resources 15. Cloud resources (e.g., cloud service systems) 15 may provide a broad range of services, including using autonomous agents (for example, bots) for monitoring patient data received from patient device 18 and, at times, responding to patient data by sending a message or control signal to patient device 18 or contacting escalation services (e.g., “911”) according to rules defined by the applicable health service policy. Autonomous agents may provide continuous (i.e., 24/7) monitoring and may trigger appropriate responses in response to any type of event (these responses could include providing a message to a patient in response to a missed dose, contacting patients in response to a trending health incident, or contacting emergency services in the event of a detected emergency, for example). Cloud resources 15 may also include databases, dynamic resource registries, health service policies, monitoring services, data analytics, on-boarding and patient activation services, and the like.

A patient device 18 may also contain health service policy logic and algorithms configured to monitor continuously a patient's health events. In certain embodiments, local health service policy logic may trigger appropriate responses to any type of event.

The care network system 10 may also illustrate a functional and communication relationship between many computing devices. However, each computing device's configurations may be governed by a patient-centered policy templates discussed below. In many embodiments, the patient-centric policy template provides a functional hierarchy and structure to the relationship of the computing devices making up the care network system 10, including providing escalation and intervention services and payer approval/preapproval services.

The devices and systems comprising the care network system 10 may be operatively coupled by one or more networks 12. Network 12 may include one or more of the Internet, local area networks (“LANs”), wide area networks (“WANs”), mobile telecommunications networks (e.g., 3G and 4G networks), and any suitable type of communication mediums. As illustrated in FIG. 1, the care network system 10 may include one or more decentralized computing/communication devices or systems (14, 15, 16, 17, 18) configured to communicate with one or more other computing devices. For example, each of a plurality of computing devices may use mobile health client software executed on a computing device using a microprocessor, random access and fixed memory, input, and output devices, and facilitating common communication protocols such as Bluetooth, Wi-Fi and 4G and/or 5G data and voice access. Some configurations may capture and store patient data in a distributed fashion, for example, on the patient's computing device, rather than storing and accessing data on a central server or plurality of distributed servers.

Each communication device (16, 17, 18) in the decentralized network 12 may originate communications to any other communication device in the decentralized network. Thus, the decentralized network may allow for scaling of compliance and intervention tasks. For example, upon detection of a compliance event, the patient device 18 or a connected device/server may determine if the detected compliance event should be escalated to an incident such that a notification or alert should be sent to a health professional or patient representative. Thus, configurations may allow for a direct one-to-many communication notifying any of the system participants of a compliance event or other event or status. The embodiments also allow for many-to-one communications, for example, from health care providers or patient representatives to the patient. Certain configurations may also provide for direct communication between the patient communication device 18 and intervention services if the there is a determination that that level of escalation is desired, for example a healthcare professional or even 911.

In addition to the patient side solutions for the patient and his/her representatives, some configurations of the disclosure may employ a decentralized patient network operations console (not shown in FIG. 1) for healthcare providers as well as for the non-professional responsible for managing multiple patients. A console/dashboard may be dynamic and driven by functional role, permissions and availability of resource enabling real-time monitoring, intervention, and mobilization of resources in response to any health event/status/escalation. This type of console functionality may enable a healthcare professional (physician, nurse, nurse practitioner, nutritionist, an allied health professional, and/or a health coach) to have a personalized dashboard specific to their role and expertise so they may provide continuity of care services concurrently to a number of members each with their own discrete, individually tailored physician directed parameters of therapeutic care.

Configurations contemplate the use of sensors that determine vitals, wellness, or other health-relevant information about a patient. Examples of these types of sensors are thermometers, scales, blood oxygen sensors, blood pressure cuffs, and similar devices that provide telemetry/biotelemetry readings or any therapeutically relevant status or reading related to a patient. The patient's computing device may operate as a sensor or may receive data from the sensors in real-time, near real-time, periodically, or event-based, when a sensor reading or measured trend passes a threshold, etc. Certain configurations may use standard (e.g., cross-platform (e.g., iOS, Android, BlackBerry OS, Windows Phone 7, etc.)) plug-ins to interact with devices. Additionally, configurations may be deployed using existing or to-be-developed computing systems and communication platforms.

Medical-related organizations may be onboarded to configurations of the system either by invitation from the system administrator or by enrolling in a program associated with the system. Patients may be onboarded in any manner for onboarding individuals to software resources. For example, they may be onboarded to the care network system 10 and become network participants using a link or invitation provided, for example, by a healthcare provider that already is a participant. A patient representative may be similarly onboarded or alternatively receive a link or invite from the patient and facilitated by the patient's mobile application. Patient representatives may be designated with one or more particular roles, thereby defining their capabilities in the system.

Configurations of the disclosure contemplate the handling of patient data consistent with government regulations (e.g., HIPPA) and patient privacy expectations. In most configurations, all patient health information (PHI) is protected by both access restrictions and encryption. For certain configurations, the access limitations are determined by the patient's permissions and/or by an associated healthcare provider. Encryption or other data protection techniques are employed relative to best practices and expected to evolve over time.

In some configurations, all data in the system is maintained in one or more logical location, such as a database, designated or agreed by the system administrator. Alternatively, more sensitive data may be maintained closer to and in storage controlled by the data owner (e.g., healthcare provider or patient), with less sensitive data flowing to a centralized logical database or server.

Configurations of the disclosure provide for access to data being designated based upon one or more of a per datum basis, a per patient basis, a per provider basis, a per publisher basis, a per subscriber basis or otherwise. In one or more configurations, multiple entities may have access to the patient data or portions thereof. For example, for a certain patient policy template, the patient data may be shared with multiple providers engaged in the treatment of the patient; perhaps a primary care physician, a medical oncologist, a surgeon, and a physical therapist. In some configurations, entities that are not providers may also have access to the data or portions thereof, such as family members, caregivers, insurers, researchers, system developers, health networks, institutions, or organizations.

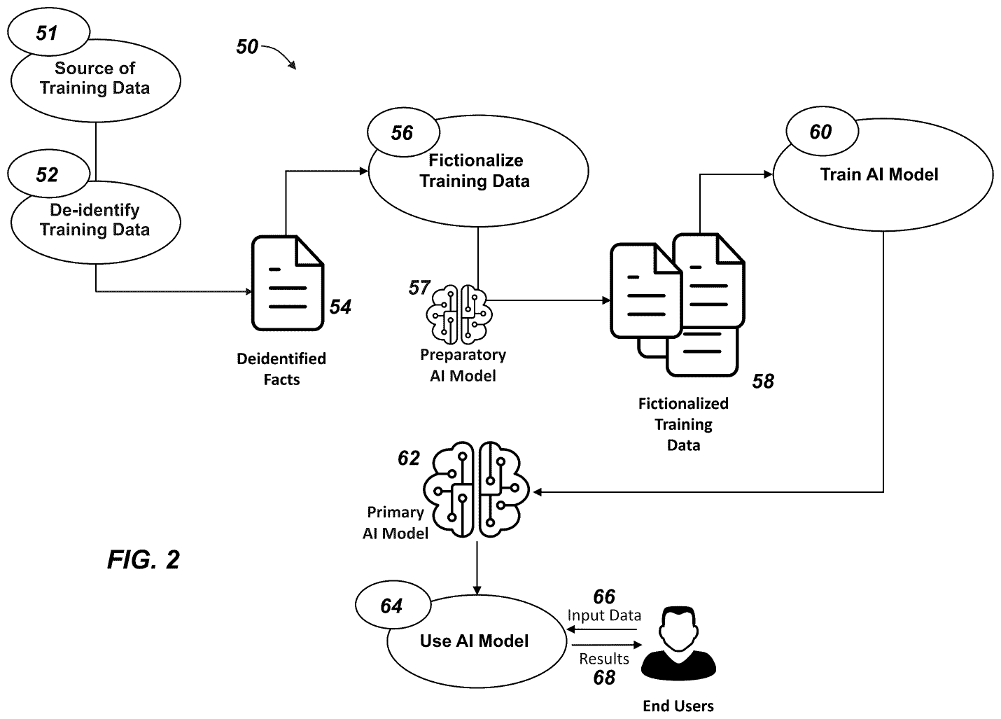

FIG. 2 illustrates a computerized system 50 in which an artificial intelligence (AI) system (i.e., an AI model 62) is made available to end users, which can provide input data to the AI model 62 and obtain results therefrom. This computerized system 50 may be part of or connected to the care network system 10 of FIG. 1. Accordingly, this computerized system 50 can be implemented using any of the various hardware, software, and other components disclosed herein.

The computerized system 50 is configured to alter patient health information in medical data used for training the AI model 62. Training data from a source 51 having patient medical information goes through de-identification steps 52 in which certain fact vectors (i.e., protected health information) are removed in computerized system 50 to protect the identity of individuals within the training data to produce de-identified training data 54 with de-identified facts. The personally identifiable information (e.g., name, DOB, etc.), which has been removed from the de-identified training data 54, is replaced by an identifier that consistently represents the same anonymous individual.

Fictionalization steps 56 in the computerized system 50 then fictionalize the de-identified training data 54 that has had de-identified facts. The fictionalization steps 56 described here is complementary to the de-identification steps 54 and adds additional identity obfuscation. This produces fictionalized training dataset 58 available to train an artificial intelligence (AI) model 62 in AI model training steps 60.

The AI model training steps 60 uses the de-identified and fictionalized training dataset 58 to train a primary AI model 62 so end users can ultimately utilize 64 the primary AI model 62 with their own input data 66 to obtain results 68. Training the primary AI model 62 is an iterative process, and corpuses of data in the training dataset 58 are commonly expanded over time. The AI protection described herein is performed as part of training data preparation, prior to each training cycle of the primary AI model 62.

To provide further AI protection, a preparatory AI model 57 can be used in the computerized system 50 to prepare the training dataset 58, which is then used in training the primary AI model 62 for access by the end user. As shown here, this preparatory AI model 57 can be used at least the fictionalization steps 56 to prepare the training dataset 58. In other arrangements, this preparatory AI model 57 can be used in either one or both of the de-identification steps 52 and the fictionalization steps 56 to prepare the training dataset 58 for use in training the primary AI model 62.

As detailed above, the technique of preparing medical data for use as training data in training the primary AI model 62 involves removing identifiable fact vectors and further fictionalizing the identifiers in the protected health information for patients whose information is to be used. The computerized system 50 seeks to preserve the integrity of certain aspects of the medical events used in training the primary AI model 62 in the AI system while sacrificing accuracy of other less important aspects.

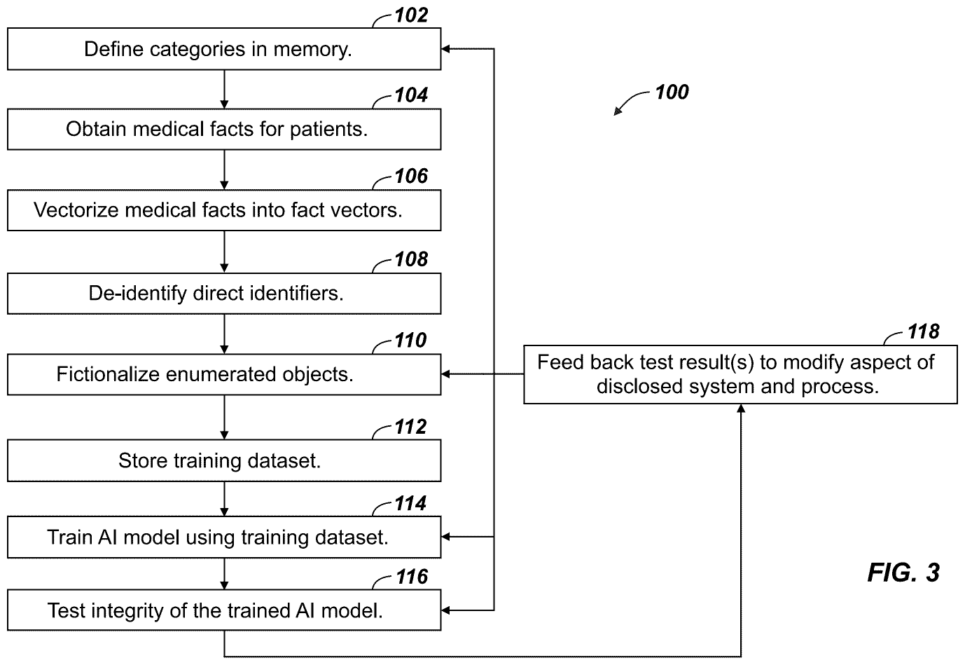

Turning now to more details of the disclosed techniques, FIG. 3 illustrates a process 100 for altering medical information so it can be used as training data to train an untrained AI model of an AI system. The process 100 is implemented using a computerized system, such as discussed with reference to FIGS. 2, 5, 6 and 7.

The computerized system (50) initiates the process 100 by defining categories in memory (Block 102). The defined categories include one or more identifier categories and one or more enumerated categories. The identifier categories serve to classify direct identifiers of personally identifiable information, while the enumerated categories categorize objects related to personally identifiable information but distinct from direct identifiers.

Also, the category parameters can be ranked from “most essential” to “least essential” as to the degree of essentialness to the generated data. Such a ranking can allow the algorithm at Block 110 to maintain more essential and true correspondences and allow fictional correspondences to vary.

The identifier categories can include pre-defined identifiers-namely, the eighteen HIPAA identifiers: a patient name, a geographical element (such as a street address, city, county, or zip code); dates related to the health or identity of individuals (including birthdates, date of admission, date of discharge, date of death, or exact age of a patient older than 89); telephone numbers; fax numbers, e-mail addresses; social security number; medical record numbers; health insurance beneficiary numbers; account numbers; certificate/license numbers; vehicle identifiers; device attributes or serial numbers; digital identifiers, such as website URLs; IP addresses; biometric elements, including finger, retinal, and voiceprints; full face photographic images; and other identifying numbers or codes. Additional categories and fields can be used depending on the nature of the medical facts.

The one or more enumerated categories subject to fictionalization can include geographic information (street address, city, state, zip code, county, census tract other than patient, medical facility, etc. as in direct identifier), temporal information (other than in a direct identifier), employment information, education information, socioeconomic information, the identity of physicians and other care takers, and other contextual information. The enumerated categories can include quasi-identifiers, which are attributes that, when combined, may potentially identify an individual. Some examples of quasi-identifiers discussed in more detail below include age, gender, zip code, and in medical contexts, certain diagnostic codes, or prescription information.

The computerized system (50) accesses and retrieves medical facts for multiple patients stored in its memory (Block 104). Using one or more processors, the computerized system (50) then transforms these medical facts into fact vectors based on the predefined categories (Block 106). At this point, the computerized system (50) then de-identifies direct identifiers in the fact vectors using de-identification steps (52) (Block 108) and fictionalizes enumerated objects in the fact vectors using fictionalization steps (56) (Block 110) to prepare a training dataset (58). Automated and manual techniques may be used to perform the de-identification and fictionalization. In one example, as noted previously, at least the fictionalization steps (56) can be performed using a preparatory AI model (57), and an expanded arrangement can use the preparatory AI model (57) to perform the de-identification steps (52) and the fictionalization steps (56).

In the de-identification steps (52), the computerized system (50) removes or alters any direct identifiers present in the fact vectors within the identifier categories (Block 108). This may involve actions such as redacting, replacing, or abstracting the direct identifiers to ensure anonymity.

For example, to de-identify any given one or more of the direct identifiers for the fact vectors in the one or more identifier categories, the de-identification steps (54) can replace the direct identifier with: a de-identifier of the given direct identifier, a code being non-descriptive of the given direct identifier, an abstraction of the given direct identifier, a substitution for the given direct identifier, a truncation of the given direct identifier, a generalization of the given direct identifier, a cryptographic hash generated from the given direct identifier, and an object encrypted from the given direct identifier.

Subsequently, in fictionalization steps (56), the computerized system (50) manipulates enumerated objects within the enumerated categories to further anonymize the data (Block 110). Actions may include swapping, shuffling, or perturbing the enumerated objects to introduce variability while maintaining the integrity of the dataset.

For example, the steps (56) of fictionalizing enumerated objects within the fact vectors can involve various techniques aimed at enhancing data privacy and anonymization. These steps (56) may include swapping enumerated objects within the fact vectors with each other, a strategy applied in at least a subset of the medical facts. Additionally, shuffling the order of enumerated objects within the fact vectors can be employed, introducing randomness and variability, particularly in a subset of the medical facts, while maintaining population-level statistical value. Another approach involves randomly reordering the enumerated objects within the fact vectors, further contributing to the obfuscation of sensitive information across a subset of the medical data.

Furthermore, the fictionalization steps (56) can incorporate the repetition of specific enumerated objects within the fact vectors throughout a subset of the medical facts, amplifying the diversity and complexity of the dataset. Moreover, the fictionalization steps (56) can include the replacement of enumerated objects within the fact vectors with abstractions, substitutions, or generalizations of the original objects. This substitution mechanism aids in minimizing the risk of re-identification while preserving the overall structure and integrity of the dataset. Overall, these techniques collectively contribute to the robust fictionalization process implemented within the enumerated categories, ensuring the confidentiality and privacy of the medical information involved.

In further examples, the steps (56) of fictionalizing enumerated objects within the fact vectors across one or more enumerated categories can include perturbing the given enumerated object within the fact vector, introducing subtle variations to the data to prevent easy identification. Additionally, the fictionalization steps (56) can employ truncation of the given enumerated object within the fact vector, reducing its length or detail to obscure sensitive information.

Furthermore, the fictionalization steps (56) can involve truncating a date associated with the given enumerated object, limiting the precision of temporal data to protect individual privacy. Moreover, adjustments to the given enumerated object by an increment can be made, modifying its value slightly while maintaining its overall context within the dataset. Similarly, numerical values of the given enumerated object can be adjusted within a predefined range, ensuring data integrity while anonymizing individual entries.

In other variations of the fictionalization steps (56), details of the medications for a patient's prescriptions may be replaced/substituted with random, benign, or other fictionalized details. Additionally, because unique or rare medical conditions can make individuals more easily identifiable, even in deidentified datasets, alternative medical diagnosis may be designated for these unique or rare medical conditions in the fictionalization steps (56).

For instance, prescription records pose a particular risk to patient privacy due to their potential to uniquely identify individuals. This vulnerability arises because most patients, especially those with chronic conditions or complex health profiles, are typically treated for multiple medical conditions simultaneously. Therefore, each patient's unique combination of medications can potentially serve as a distinctive identifier, much like a fingerprint. Moreover, when combined with even limited demographic data (like age range or gender), prescription data becomes even more potent for reidentification.

For this reason, the risk of reidentification through medication profiles is particularly acute. For example, certain combinations of medications are highly specific to particular conditions or disease states so that an individual's combination of prescriptions can be highly specific, especially for those with multiple or rare conditions. For instance, a patient taking both an antiretroviral and an antidepressant might be easily identifiable in a small community. Some patients require uncommon or highly specialized drugs, which can make their medical profile stand out even in larger datasets.

The timing and frequency of prescription refills create a unique pattern that can serve as a fingerprint for identification. The timing of prescription changes or additions can provide additional identifying information, especially for patients with evolving health conditions. When dosage information is included, the specific dosages can further narrow down a pool of potential individuals, especially for medications that are carefully titrated.

To mitigate these risks, the fictionalization steps (56) can employ several strategies, such as drug class substitution, generic descriptions, grouping, removal, aggregation, and generalization, in the fictionalization process. Instead of listing specific medications, the fictionalization steps (56) can substitute equivalent drug classes. This approach can preserve the general nature of the treatment while obscuring the exact medication. For example, “GLP-1 receptor agonist” can be used instead of “Ozempic” (semaglutide). Nevertheless, dosage information, especially for weight-based medications, can provide clues about an individual's physical characteristics.

The fictionalization steps (56) can use broad, functional descriptions of medications. For instance, “blood pressure medication” can replace specific antihypertensive drug names. The fictionalization steps (56) can remove certain medications, which may be extremely rare or identifying medications, and can replacing them with a general placeholder like “specialized medication.” Dosage information can be aggregated or listed in broader categories, such as “low dose” or “high dose.”

Even if a prescriber's name is removed, the combination of medications may be indicative of a specific specialist or clinic, narrowing down the patient pool. The disclosed fictionalization process can generate equivalent real prescribers for the fictionalized data. Information about where prescriptions are filled can provide geographical clues about a patient's residence or workplace. The fictionalization steps (56) can generate equivalent real pharmacy locations for the fictionalized data.

Insurance reimbursements can pose a significant de-identification challenge that increases the risk of re-identification. Prescription codes, such as National Drug Codes (NDCs) or other standardized codes, used in prescriptions can provide extremely specific information about the exact medication, strength, and form prescribed. The fictionalization steps (56) can fictionalize these details.

Related conditions can be grouped together. For example, rather than listing individual medications for related conditions, a broader category may be used. For example, “cardiovascular medications” can encompass drugs for hypertension, high cholesterol, and heart rhythm disorders. Long-term prescriptions for chronic conditions create persistent patterns that need to be anonymized effectively. Finally, temporal data can be generalized. Exact dates of prescription changes can be replaced with broader time frames, such as seasons or quarters.

Another aspect of the fictionalization steps (56) includes shifting the date associated with the given enumerated object, introducing temporal displacement to mitigate the risk of re-identification. Lastly, the fictionalization steps (56) can use averaging of the given enumerated objects within the fact vectors, particularly in a subset of the medical facts. This averaging mechanism contributes to data anonymization by obscuring individual values and promoting statistical consistency across the dataset. Collectively, these techniques serve to safeguard the privacy of sensitive medical information while preserving the utility and integrity of the dataset.

Overall, the medical facts can be clustered into clusters based on the fact factors in two or more of the categories. In this way, fictionalizing the given enumerated objects for the fact vectors in each of the clusters can use a same form of the fictionalization process. For example, the identity of the treating physician can be fictionalized in the same way for all facts of that cluster pertaining to that patient/physician relationship. Because possible fictionalization may be done through the introduction of a synthetic physician's identity for every physician/patient relationship, this weakens physician identity as a hacking vector in possible triangulated attacks.

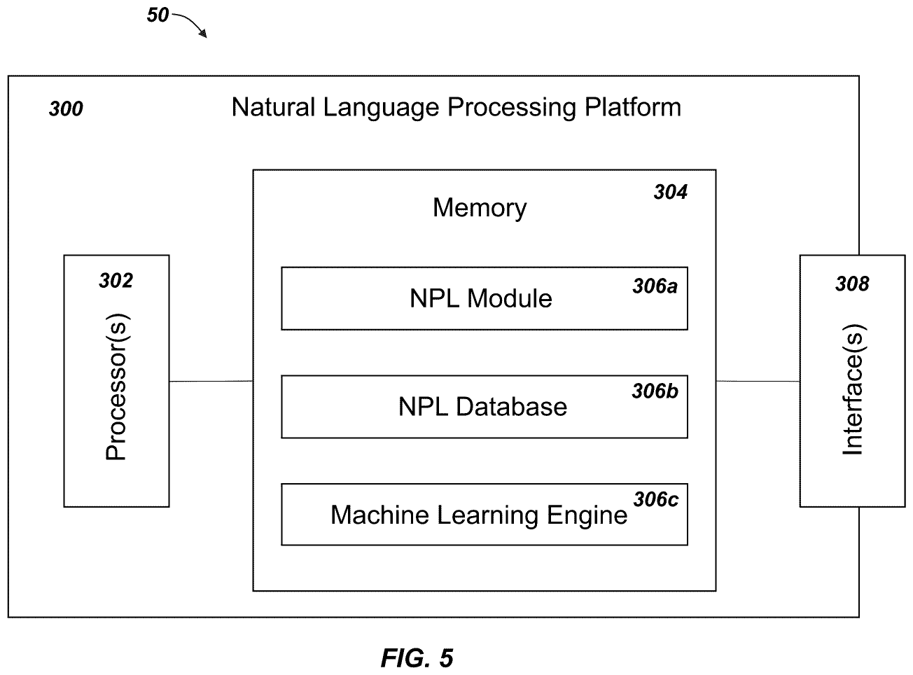

The steps of vectorizing (Block 106), de-identifying (Block 108), and fictionalizing (Block 110) can use a natural language processing platform of the computerized system, such as discussed below with reference to FIG. 5. After vectorization, de-identification, and fictionalization, the computerized system in the disclosed process 100 stores the modified medical facts in memory as a training dataset (Block 112).

Utilizing this training dataset, the computerized system (50) proceeds to train an untrained artificial intelligence (AI) model (Block 114). For example, through repeated processing of the de-identified and fictionalized training dataset (58) and adjustment of weights within a neural network training framework, the computerized system (50) guides the primary AI model (62) to converge towards desired outputs. These active training steps (60) empower the computerized system (50) to transform the initially untrained AI model into a trained AI model (62) capable of performing tasks relevant to the medical dataset and the underlying purpose of the AI model (62).

Additionally, the process 100 may involve testing the trained AI model (62) to assess its performance and reliability (Block 116). This testing phase typically includes probing the AI model (62) with public facts associated with the medical dataset to evaluate its ability to handle real-world scenarios effectively. Through these iterative steps, the computerized system ensures the robustness and effectiveness of the AI model in handling medical data while preserving privacy and confidentiality.

Finally, the process 100 may involve feeding back test result(s) from the testing to modify an aspect to the disclosed system and processes (Block 118). For example, an aspect of at least one of: the enumerated categories defined to be fictionalized (Block 102); the fictionalization steps (56) used to fictionalize the enumerated objects (Block 110); the training steps (60) used to train the AI model (62) (Block 114); and the testing steps used to test the AI model (62) (Block 116) can be modified in the systems and processes disclosed herein based on a result from the testing of the trained AI model (62). These and other aspects can be modified.

As noted above, the de-identification and fictionalization steps (52, 56) in the process 100 seek to remove and/or obscure personal identifiers from health records to protect patient privacy while still preserving the data's utility for training the primary AI model (62). As will be appreciated from the above discussion, implementing these strategies for the de-identification and fictionalization steps (52, 56) needs to balance the remaining utility of the resultant training dataset (58) and the amount of privacy protection offered.

The level of generalization or substitution can be tailored to a specific context, considering factors such as the size of the training dataset (58), the rarity of the conditions involved, and the intended use of the de-identified facts and fictionalized training data. Clinically relevant relationships between variables are preferably preserved so that important data is not overly suppressed. Of course, the disclosed process 100 of altering patient health information in medical data used for training an AI model (62) can be part of a comprehensive approach that uses additional safeguards, such as strict data access controls, user agreements, and ongoing risk assessments, to protect patient privacy in medical research and data sharing.

As expected, removing too much information can make the data less useful for training an AI model (62), while removing too little risks patient re-identification. Medical records contain various data types (e.g., demographics, diagnoses, treatments, lab results), and each data type presents unique challenges for deidentification and fictionalization.

Health records often span long periods, which can increase the risk of reidentification through data aggregation over time. By adjusting the time scale of fictionalized noncritical conditions, the fictionalization steps (56) can obscure the true data.

Advances in data mining and artificial intelligence are being encountered as technology progresses. De-identified data may become vulnerable to re-identification through sophisticated analysis techniques. To illustrate the power of prescription data for re-identification, a study published in the Journal of the American Medical Informatics Association in 2013 found that knowing an individual's year of birth, gender, and the state in which they lived, along with just two to three prescription medications, was enough to uniquely identify over 60% of individuals in a large prescription dataset.

Given these challenges, the de-identification and fictionalization steps (52, 56) of the present disclosure can perform more than simply removing direct identifiers for prescription data to be used in the training dataset (58) of the AI model (62).

In particular, K-anonymity is a property of an anonymized dataset that helps quantify the degree of protection afforded to individuals in that dataset. K-anonymity can be used in the fictionalization steps (56) to ensure that each combination of quasi-identifiers applies to at least K individuals in the training dataset (58). The training dataset (58) can then be said to have K-anonymity if, for every combination of quasi-identifiers in the dataset, there are at least K individuals who share those exact quasi-identifier values.

In the K-anonymity technique, the quasi-identifier noted previously is an attribute that, when combined, could potentially identify an individual patient. Examples of quasi-identifiers can include, but are not limited to, age, gender, zip code, and certain diagnostic codes or prescription information in medical contexts. The K value in the K-anonymity technique is the minimum number of individuals that must share the same combination of quasi-identifiers. A higher K-value generally indicates stronger anonymization.

In the K-anonymity technique, the quasi-identifiers are identified by determining which attributes in the dataset can potentially be used for identification. Grouping is performed to group records with similar quasi-identifier values together.

Generalization or suppression steps are then applied to ensure each group contains at least K individuals. In the generalization step, the range of values (e.g., exact age to age range) are broadened. In the suppression step, certain values are removed or masked. Finally, a verification step can confirm that, for every combination of quasi-identifiers, there are at least k individuals with those exact values.

As a simple example, a medical dataset may have the quasi-identifiers: Age, Gender, and Zip Code.

The original data can be as follows:

| Age | Gender | Zip Code | Disease |
|---|---|---|---|
| 28 | M | 12345 | Flu |
| 31 | F | 12345 | Diabetes |
| 28 | M | 12346 | Hypertension |
| 35 | F | 12345 | Asthma |

To achieve 2-anonymity, the data can be generalized as follows:

| Age | Gender | Zip Code | Disease |
|---|---|---|---|
| 25-35 | M | 1234* | Flu |
| 25-35 | F | 1234* | Diabetes |
| 25-35 | M | 1234* | Hypertension |
| 25-35 | F | 1234* | Asthma |

Now, for each combination of quasi-identifiers, there are at least two individuals and preferably multiple individuals within the dataset.

Achieving K-anonymity may require generalizing or suppressing data, which can reduce its utility for analysis. If all K individuals in a group have the same sensitive attribute, an attacker can still infer that attribute for any individual in the group. External information can sometimes be used to narrow down possibilities within a K-anonymous group. As the number of quasi-identifiers increases, achieving K-anonymity becomes more challenging and may require more aggressive generalization or suppression.

To address some limitations of using K-anonymity in the fictionalization steps (56), other techniques can be used, such as L-diversity or T-closeness techniques. The L-diversity technique ensures diversity in the sensitive attributes within each group. The T-closeness technique requires the distribution of sensitive attributes in any group to be close to their distribution in the whole dataset.

When applying anonymization techniques, such as the K-anonymity technique discussed above, there is an inherent risk of introducing distortions or errors into the training dataset (58). These distortions can potentially lead to incorrect conclusions or biased analyses when training the AI model (62). Several strategies can be used to minimize distortion and errors.

First, careful selection can be made of the quasi-identifiers. Only the necessary attributes may be identified as quasi-identifiers. Also, the number of quasi-identifiers can be reduced to minimize the need for generalization.

As noted above, the quasi-identifier is an attribute (or a set of attributes) in a dataset that, while not directly identifying an individual, can be combined with other information to potentially identify a specific individual. The quasi-identifiers are not unique to an individual, unlike direct identifiers (e.g., Social Security numbers, etc.). However, the quasi-identifiers have special combinatorial power. When combined with other quasi-identifiers or external information, they can lead to unintended re-identification. In that sense, the quasi-identifiers are context dependent. What constitutes a quasi-identifier can vary based on the dataset and external available information and can vary based on the dataset's intended usage. For example, enrollment in a clinical trial or other special group may be a quasi-identifier in some contexts but not others.

Generalization hierarchies can be designed to preserve as much information as possible. Domain experts can review the anonymized data to ensure that important relationships and patterns in the data are preserved. In this way, the domain expertise can ensure that the generalizations make sense in the context of the data.

Under a minimal distortion principle, the least amount of generalization or suppression can be applied as necessary to achieve K-anonymity. Optimization algorithms can find the minimal distortion that satisfies privacy requirements. Accordingly, the disclosed systems and processes can seek to maintain key statistical properties (mean, variance, correlations) after anonymization.

In the end, a number of considerations can be used to evaluate the fictionalization steps (56) of the medical data. The considerations can include one or more of: (i) verifying that the fictionalization steps (56) have preserved clinically relevant relationships between the medical facts; (2) verifying that the fictionalization steps (56) has not overly suppressed rare but important medical conditions; (3) verifying that the fictionalization steps (56) has maintained the integrity of time-series data; (4) verifying that the fictionalization steps (56) has maintained special and generalized dosage information in prescription data; (5) verifying that the fictionalization steps (56) has maintained data integrity while anonymizing; etc. If any of these verifications are not true, the parameters for the fictionalization steps (56) can be adjusted and reapplied.

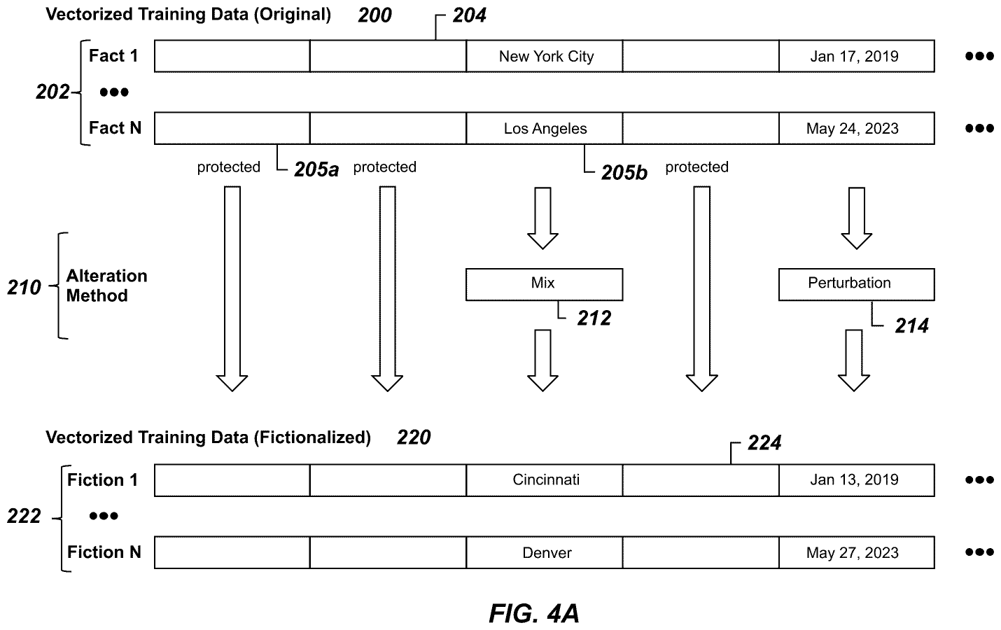

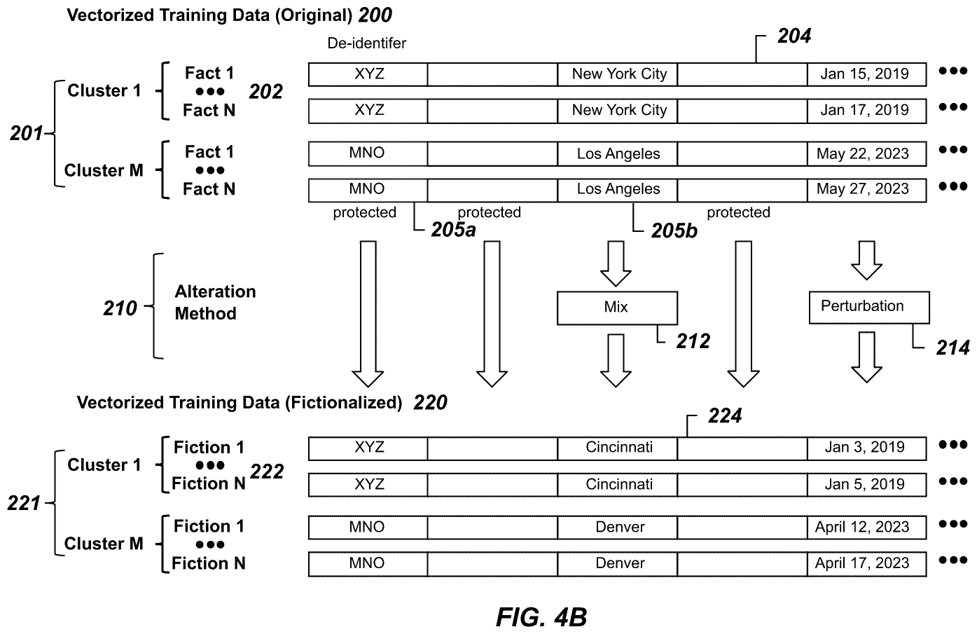

FIG. 4A illustrates a diagram of altering medical facts found in medical data to be used in training AI models in AI systems. As shown in FIG. 4A, training data 200 includes a list of medical facts 202 being prepared so they can be used in training data to train an AI model. The medical facts 202 are first vectorized (i.e., broken out into an array of fields 204). The medical facts 202 can be categorized into various fields or vectors 204 that depend on the medical facts being used and their purpose.

As noted, the systems and processes disclosed herein first de-identify any identifiers in certain fact vectors by replacing them with de-identifiers as shown. Then, the systems and processes apply alteration method(s) 210 fictionalize specified fact vectors 205b in the training data 200. The identifiers in the fact vectors 205a are removed to protect the identity of individuals within the training data. In the de-identification, the personally identifiable information (i.e., having an identifier such as name, DOB, etc.) is replaced by a de-identifier that consistently represents the same anonymous individual.

In general, the vectorized fact vectors 205a having personally identifiable information (e.g., name, DOB, etc.) can be categorized according to identifiers-namely, the eighteen HIPAA identifiers previously detailed.

Initially, the fact vectors 205a with these enumerated identifiers are protected by de-identification, and the de-identifiers of these fact vectors 205a remain protected (unaltered) in the alteration method(s). Other fact vectors 205b having enumerated details or categories are altered by the disclosed fictionalization steps 212, 214 of the alteration method(s) 210, and the resulting vectorized/fictionalized training data 220 can be used for training an AI model for an AI system. To perform the fictionalization steps 212, 214, the systems and processes determine which fact vectors 205a having de-identifiers are protected and what alteration methods 210 (e.g., steps 212, 214) will be applied to alter each of the unprotected fact vectors 205b.

In one approach, the fictionalization step 212 uses a mixing technique in which the values of a given fact vector 205b for each medical fact 202 are treated as a set of items, which can be randomly shuffled and redistributed among the other medical facts 202. The mixing technique 212 retains the statistical nature of the values of this field of fact vector 205b while obscuring the individual facts. For example, the mixing technique 212 can be used to redistribute a geographical element, such as the location of the medical fact vector.

In another approach, the values of a fact vector 205b can be perturbed within a range using a perturbation technique 214. For example, date-related information can be perturbed within a range (e.g., by moving dates within +/−5 days or so). This also retains some accuracy while obscuring the individual facts.

Other alteration methods 210, such as substituting a fact vector with a synthesized identifier, like substituting a physician/patient relationship for a single physician's identity, are also possible, as described above.

The alteration methods 210 of given fact vectors 205b produces a resulting vectorized/fictionalized training data 220, in which identifier fact vectors 205a have been de-identified and in which auxiliary fact vectors 205b have been fictionalized. The resulting vectorized/fictionalized training data 220 that comes from applying these techniques may not have all of the utility of one built upon unaltered medical facts. The choices made for which fact vectors 205a to protect and which fact vectors 205b to alter are configured to the expectations of the AI model's application.

The fictionalization performed by the alteration methods 210 can be iteratively performed. For example, the training data 200 with the list of medical facts 202 can be de-identified and can be subjected to more conservative forms of fictionalization by the alteration methods 210 in first iterations. In later iterations, the constraints for the alteration methods 210 can be relaxed. The anonymized data in the resulting vectorized/fictionalized training data 220 can then be tested at each of the iterations. Then, based on the different results of the iterations, an optimal balance can be found between providing anonymity and maintaining data utility.

In testing the resulting vectorized/fictionalized training data 220, metrics can be employed to quantify information loss, such as a discernibility metric that measures how many records are indistinguishable from each other. Error bounds and confidence intervals can be used with the anonymized data. These can help define any potential range of distortion. Statistical tests can be performed before and after anonymization to detect significant changes. Finally, a portion of the training data 200 can be used as a validation set to check for distortions in the vectorized/fictionalized training data 220.

FIG. 4B diagrams a process of altering clusters 201 of medical information found in medical data to be used in training AI systems. As shown, training data 200 includes a list of medical facts 202. Again, the medical facts 202 are first vectorized (i.e., broken out into an array of fields 204). The medical facts 202 can be categorized into various fields or vectors 204 that depend on the medical facts being used and their purpose. Then, the system de-identifies any identifiers in certain fact vectors 205a by replacing them with de-identifiers as shown. Next, the medical facts 202 are clustered together in clusters 201. For example, the clusters can group the medical facts according to one or more criteria, such as a de-identifier (e.g., the person who has been de-identified); a medical episode (e.g., a cluster of common medical facts in a single episode); and a combination of fields (e.g., a physician/patient relationship).

The clustering can be performed to achieve the K-anonymity technique, L-diversity technique, T-closeness technique, or the like, as discussed previously. Additionally, micro-aggregation can be used to preserve certain statistical characteristics. The micro-aggregation is a statistical control technique used to protect the privacy of individuals in a dataset. In micro-aggregation, the dataset is partitioned into small groups (or clusters) of similar records, and the original values are replaced with aggregate values for each group. As a basic example, age ranges can be grouped in 10-year spans (0-10, 10-20, 20-30, etc.). For instance, patients' medical records can be aggregated as simply pediatric (0-18), young adult (18-39), middle age (40-64), young-old (65-74), middle-old (75-84), and old-old (85+).

After these steps, the disclosed systems and processes apply alteration method(s) 210 fictionalize specified fact vectors 205b in the training data 200. The alteration method(s) 210 shown here can include a mixing technique 212 and a perturbation technique 214 described above. The alteration method(s) 210 are applied uniformly to all the medical facts 202 in a cluster 201 to retain the medical episode's factual accuracy when fictionalized. For example, a medical episode may cover the combined symptoms surrounding a chemotherapy infusion treatment. The alteration method(s) 210 retain the order of the medical facts 202, while moving the entire episode in space and time during the fictionalization to obscure the identity of the person.

As noted above, the alteration methods 210 include a mixing technique 212 and a perturbation technique 214. Other alteration methods can be used to fictionalize information.

In one form of geographic alteration, geographic fictionalization can be used to fictionalize geographic-related facts. The geographic fictionalization can be used to fictionalize anything that indicates a location of an event. “Location” can include postal address fields, the names of hospitals, and the like. When the locations of facts are altered, the fictionalized facts produced in the resulting change are preferably plausible. For example, changing the location (address) from a hospital to some random location (address of a gas station) would result in an implausible statement that could yield an exploitable vulnerability of the protected information.

In another form of geographic alteration, geographic mixing can be used to fictionalize geographic-related facts. From the fact set, geographic mixing extracts the list of geographic locations, shuffles the list, and picks a geographic location from the list, without replacement. This process applies a new geographic location to each fact or fact cluster.

In yet another form of geographic alteration, geographic permutation can be used to fictionalize geographic-related facts. For each given fact or fact cluster, geographic permutation forms a list of possible locations within a specified radius, area, or region. Geographic permutations choses a random location from this list and applies the selection to the given fact or fact cluster.

Temporal-related information can also be altered. In one form of temporal alteration, temporal fictionalization fictionalizes anything that indicates when an event occurred. These can include dates, times, seasons, etc. When the time of facts are altered, the resulting change is preferably plausible. For example, the time that a patient reports a fact about his/her condition should be after the condition diagnosis.

In another form of temporal alteration, temporal mixing extracts the list of dates/times from a fact set, shuffles the list, and picks a date/time from the list, without replacement. This alteration applies a new date/time to each fact or fact cluster.

In yet another form of temporal alteration, temporal permutation forms a list of possible date/times within a time window for each given fact or fact cluster. A random date/time is chosen from this list and applies the selection to the given fact or fact cluster.

As noted, the fictionalization preferably maintains plausibility and consistency with the underlying facts. Maintaining plausibility for an alteration requires an understanding of the fact stream's structure and the semantic interrelatedness of different fact classes. For example, a patient reporting his/her symptoms that resulted from a medication side effect must occur after the medication was taken. A data scientist, or a specifically trained AI model that understands these causal relationships, can direct the construction of the alteration methods prior to modifying the training facts.

Several types of AI models can be used for the techniques of the present disclosure, including, for example, a symbolic AI model, a Bayesian network model, a machine learning model, a neural network model, and types of algorithmic models. A few of these models are briefly described.

As is known, a Bayesian network (a.k.a. a belief network or a causal probabilistic network) is a graphical model representing probabilistic relationships among a set of variables. The model includes nodes (representing variables) and includes directed edges (representing probabilistic dependencies) between the variables. At its core, the Bayesian network provides a joint probability distribution of the variables in a compact form by employing conditional independence relationships.

Each node in the Bayesian network represents a random variable and is associated with a conditional probability distribution that quantifies the probability of that variable given its parent variables. The parent variables of a node are the variables that directly influence the node's probability distribution. By specifying these conditional probabilities for each node, the Bayesian network encodes the entire joint probability distribution of the variables.

For their part, the directed edges in the Bayesian network define causal relationships or dependencies among variables. The direction of the edges indicates the direction of influence: from parent variables to child variables. This structure enables efficient inference by allowing reasoning about the probabilities of variables given observed evidence or interventions on certain variables.

A symbolic AI model uses explicit rules and logical reasoning to solve problems. In the symbolic AI model, knowledge is represented in a structured format, such as rules or ontologies, and algorithms manipulate these rules to derive conclusions or make decisions. The knowledge can be encoded as rules or if-then statements to provide recommendations or solutions.

A machine learning model learns from data to improve performance over time without being explicitly programmed. Several subtypes, including supervised learning, unsupervised learning, and reinforcement learning, can be used. Supervised learning involves training a model using labeled data, where the input-output relationships are explicitly provided. Supervised learning is suitable for classification and regression types of tasks. Unsupervised learning trains models using unlabeled data to discover hidden patterns or structures within the data and is suitable for clustering and dimensionality reduction. In reinforcement learning, an agent learns to make sequential decisions by interacting with an environment and receiving feedback in the form of rewards or penalties.

A neural network model has interconnected nodes, or neurons, organized into layers. Each neuron applies a mathematical operation to its inputs and passes the result to the next layer. Deep learning as a subset of a neural network trains models with multiple layers in a deep architecture to learn hierarchical representations of the data. Convolutional neural networks (CNNs) are suitable for image recognition and computer vision, whereas recurrent neural networks (RNNs) are suited to analyzing sequential tasks, such as natural language processing and time series prediction.

An algorithmic model iteratively evolves candidate solutions using mechanisms such as mutation, crossover, and selection. The algorithmic model is useful for problems having large search spaces or complex, multi-dimensional objective functions.

As one example according to the present disclosure, FIG. 5 illustrates a natural language processing platform 300 for preparing a training dataset to be used in training an AI model in a computing environment. This NLP platform 300 can prepare a primary training dataset (58) in the training of a primary AI model (62) in the computerized system (50) of FIG. 2. Moreover, this NLP platform 300 can prepare a preparatory training dataset in the training of a preparatory AI model (57) in the computerized system (50) of FIG. 2.

The NLP platform 300 can use large language models (LLMs) to enable a computing system to understand, process, and generate language and perform tasks that construct, arrange, and manage medical information to be used as training data for AI models. In general, the NLP platform 300 can be associated with an organization or entity (e.g., a health care company, a research entity, an insurance agency, or the like).

As disclosed herein, the NLP platform 300 can be configured to perform NLP processing techniques to prepare training data of medical information to be used to train AI models in an AI system. Additionally, the NLP platform 300 can maintain a model that the NLP platform 300 may use to prepare the training data. In particular, the NLP platform 300 can be configured to perform NLP processing techniques to de-identify and fictionalize certain medical facts for the training data used in training AI models in an AI system according to the present disclosure. Moreover, the NLP platform 300 can dynamically update the training data as additional inputs, rules, restrictions, etc. are received.

The natural language processing (NLP) platform 300 may include one or more computing devices configured to perform one or more of the functions described herein. For example, the NLP platform 300 may include one or more computers (e.g., servers, server blades, laptop computers, desktop computers, etc.).

The NLP platform 300 can include one or more processors 302, memory 304, and an interface 306. A data bus can interconnect the processor 302, the memory 304, and interface 306. In general, the interface 306 can be a network interface configured to support communication between the NLP platform 300 and one or more networks (not shown).

The memory 304 may include one or more program modules having instructions that when executed by processor 302 cause the NLP platform 300 to perform one or more functions and/or one or more databases that may store and/or otherwise maintain information which the program modules may use and/or the processor 302. In some instances, the one or more program modules and/or databases may be stored by and/or maintained in different memory units of the NLP platform 300 and/or by different computing devices that may form and/or otherwise make up the NLP platform 300. For example, the memory 304 may have, store, and/or include the NLP module 306a, an NLP database 306b, and a machine learning engine 306c.

The NLP platform 300 may have instructions that direct and/or cause the NLP platform 300 to execute advanced natural language processing techniques. For example, the NLP platform 300 can apply NLP techniques to de-identify identifiable facts and to alter (fictionalize) other facts.

The memory 304 may include several components or modules, as illustrated. The NLP database 306b may store information used by the NLP module 306a and/or the NLP platform 300 in de-identifying identifiable facts and altering (fictionalizing) other facts, and/or in performing other functions. The machine learning engine 306c may have instructions that direct and/or cause the NLP platform 300 to perform de-identification steps and fictionalization steps, and to set, define, and/or iteratively refine optimization rules and/or other parameters used by the NLP platform 300 and/or other systems.

The following section generally describes the training an untrained Deep Neural Network (DNN) to generate a trained model using training data according to the present disclosure. The neural network is trained based on the training data, which is de-identified and fictionalized by the techniques of the present disclosure.

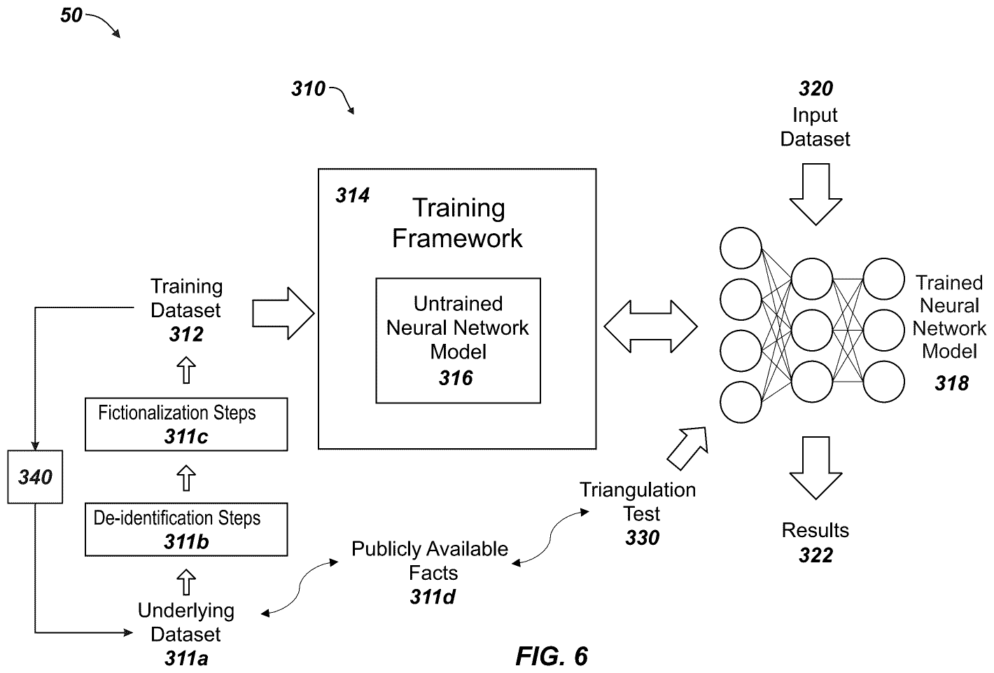

As a further example according to the present disclosure, FIG. 6 illustrates a process for training and deploying a trained neural network model 318 of a primary deep neural network 310 to process an input dataset 320 to produce results 322 according to a primary purpose of the present disclosure. The following section generally describes the training of an untrained neural network model 316 to generate the trained neural network model 318 using a training dataset 312. The training can be used to train a primary AI model (62; FIG. 2) for use by end users as described above in the computerized system (50) of FIG. 2.

The neural network 310 is structured for its task so an end user can process inputs and receive results according to the underlying purpose of the implementation. Once structured, the neural network 310 is then trained using a training dataset 312. Initially, the training dataset 312 of medical information is first processed to remove identifier facts and to fictionalize other facts (geographic information, time information, etc.) of an underlying dataset 311a using de-identification steps 311b and fictionalization steps 311c, such as described previously in the steps (52, 56; FIG. 2) and in the alteration methods (210; FIGS. 4A-4B).

As noted above, the alteration methods (210; FIGS. 4A-4B) performed in the de-identification steps 311b and the fictionalization steps 311c can be iteratively performed, as depicted by an iterative process 340 in FIG. 6. For example, the underlying dataset 311a with the list of medical facts can be de-identified and can be subjected to more conservative forms of the alteration methods of the fictionalization steps 311c in first iterations. In later iterations, the constraints for the alteration methods in the fictionalization steps 311c can be relaxed. The anonymized data in the resulting training dataset 312 can be tested at each iteration to find an optimal balance between providing anonymity and maintaining data utility.

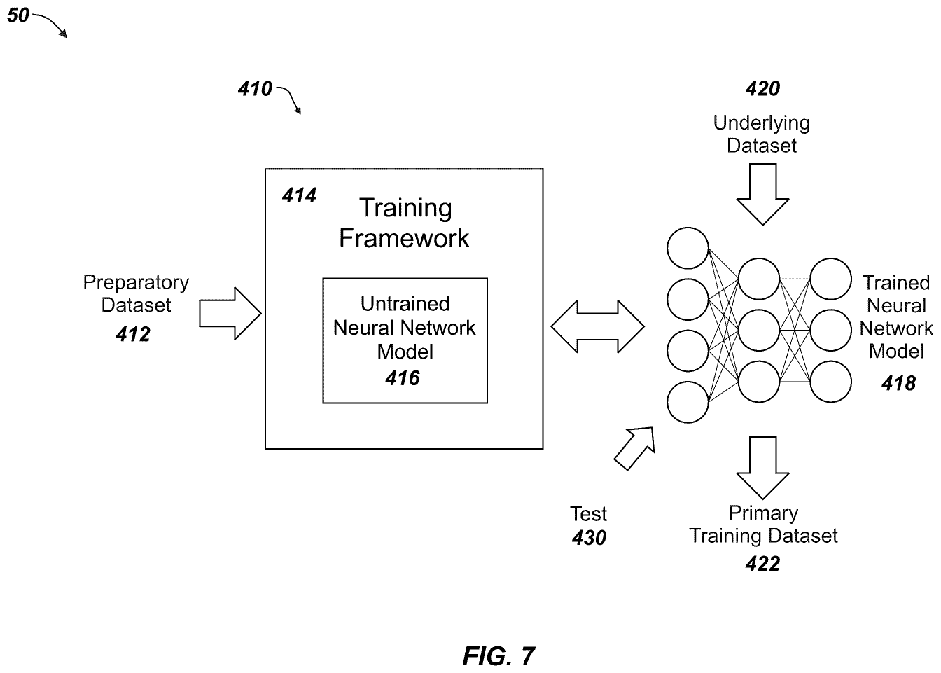

This iterative process 340 can itself use an AI training framework that trains a preparatory AI model (57; FIG. 2) to de-identify and/or fictionalize the original dataset 311a to generate synthetic data for the training dataset 312. This synthetic data produced by the preparatory AI model (57; FIG. 2) may then preserve statistical properties while providing strong guarantees of privacy. Further details of this approach are provided below with reference to FIG. 7.

In FIG. 6, once the training dataset 312 has been de-identified and fictionalized according to the techniques of the present disclosure, training of the deep neural network 310 can begin. To begin training the deep neural network 310, initial weights for the untrained model 316 may be chosen randomly or by pre-training using a deep belief network. The training framework 314 then performs a training cycle, which can then be performed in either a supervised or unsupervised manner.

Supervised learning uses the training dataset 312 to teach models to yield the desired output. The training dataset 312 includes inputs and desired outputs, which allow the model to learn over time, or when the training dataset 312 includes input having known output and the output of the neural network is manually graded.

The training framework 314 of the deep neural network 310 processes the inputs and compares the resulting outputs against a set of expected or desired outputs. Errors are then propagated back through the training framework 314. As training proceeds, the training framework 314 can adjust and change the weights that control the untrained neural network model 316. The training framework 314 can provide tools to monitor how well the untrained model 316 is converging towards a model suitable for generating correct answers based on known input data. The training process repeatedly occurs as the network weights are adjusted to refine the output generated by the neural network. The training process can continue until the neural network reaches a statistically desired accuracy associated with a trained neural network model 318. Ultimately, the trained model 318 can then be deployed in the disclosed process (100) so the trained model 318 presented with an input dataset 320 can implement the machine learning operations and output a result 322 for use according to the purposes of the disclosed process (100).

Supervised learning is typically separated into two types of problems-classification and regression. Classification uses an algorithm to assign test data accurately into specific categories. Regression is used to understand the relationship between dependent and independent variables. Numerous different algorithms and computation techniques can be used in supervised machine learning, including but not limited to, neural networks, naïve bayes, linear regression, logistic regression, support vector machines (SVM), k-nearest neighbor, and random forest.

As previously noted, unsupervised learning is a learning method in which the network uses algorithms to analyze and cluster unlabeled data. These algorithms discover hidden patterns or data groupings. Therefore, the training dataset 312 includes input data without any associated output data. The untrained neural network model 316 can learn groupings within the unlabeled input and determine how individual inputs relate to the overall dataset.

Unsupervised training can be used for three main tasks-clustering, association, and dimensionality. Clustering is a data mining technique that groups unlabeled data based on similarities and differences. This technique is often used to process raw, unclassified data objects into groups represented by structures or patterns in the information. Association is a rule-based method for finding relationships between variables in a given dataset. This method is often used for market basket analysis. Dimensionality reduction is used when a given dataset's number of features (dimensions) is too high. This technique is commonly used in the preprocessing of data.

Variations of supervised and unsupervised training may also be employed. Semi-supervised learning is a technique in which the training dataset 312 includes a mix of labeled and unlabeled data of the same distribution. Incremental learning is a variant of supervised learning in which input data is continuously used to train the model further. Incremental learning enables the trained neural network model 318 to adapt to the input dataset 320 without forgetting the knowledge instilled within the network during initial training.

Those skilled in the art will appreciate there are many technologies for machine leaning and creating artificial intelligence systems. Gradient descent is one algorithm that enables an application developer to teach a model and iteratively refine its predictions, improving accuracy in detecting medical states, such as learning the causation of adverse events based on fictionalized clinical data. Accordingly, the neural network 310 of FIG. 6 can use gradient descent in the training process of the neural network model 318.

For example, the neural network 310 may be designed to recognize adverse events in medical records. The goal is to train the neural network model 318 to identify instances where certain clinical markers suggest potential complications.

The training process begins by framing the task as an optimization problem. The untrained neural network model 316 is constructed, with the input being clinical data and the output representing probabilities of different adverse events. The network's weights are initially unknown. For each record in the training dataset 312, the untrained model 316 generates an output, which is compared to the correct classification. The difference between the predicted and actual outputs is squared and summed to produce a cost for that example. Summing up these costs across the entire dataset yields the overall cost function.

The objective is to minimize this cost by adjusting the network's weights. Gradient descent facilitates this by iteratively computing the gradient of the cost function with respect to the weights. At each iteration, the training framework 314 evaluates the current cost, computes the gradient (indicating the direction in which the cost increases), and updates the weights by taking a small step in the opposite direction. This process is repeated until the cost is minimized, or a stopping criterion is met.

There are several variants of gradient descent. Stochastic gradient descent (SGD) updates a model's weights based on a random subset of data, reducing computation time when dealing with large datasets. Adaptive gradient descent adjusts the step size for each weight independently, which is useful for handling complex or sparse medical data. Momentum gradient descent incorporates the gradient from previous steps to accelerate convergence.

As discussed herein, the goal of the primary trained neural network model 318 of FIG. 6 is to process an end user's input dataset (320) and provide results (322) that are relevant and accurate. The other goal for the neural network model 318 is to also protect and anonymize the underlying medical information that has been used in the training the neural network models 318. Thus, once the deep neural network 310 has been trained with the de-identified and fictionalized training dataset of the present disclosure, the original medical information is altered because (i) PHI has been excised and (ii) associated information has been fictionalized by being replaced with altered (mixed or perturbed) information.

The altered (mixed or perturbed) information is intended to retain some of the underlying character, nature, and context of the original information. For example, geographical information, which may be useful in terms of understanding the broader geographical context of the medical information being analyzed, may be replaced (fictionalized) in a way that retains some of the broader geographical context, while retaining some of the geographical area (e.g., city, state, region, etc.) of the original information. Likewise, temporal information, which may be useful in terms of understanding broader temporal context of the medical information being analyzed, may be replaced (fictionalized) in a way that retains some of the broader temporal context, while retaining some of the temporal details (e.g., day, month, season, time zone, etc.) of the original information.

As will be appreciated, a neural network that has been trained using sensitive medical information may inadvertently leak protected health information. The de-identification and fictionalization processes disclosed herein aim to provide differential privacy that protects individual privacy during training. Yet, the trained neural network model 318, like any other software system, can still be vulnerable to hacking. For this reason, the de-identification and fictionalization processes disclosed herein also aim to mitigate hacking attempts of the trained neural network model 318 seeking to obtain underlying facts from the medical information used to train the trained neural network model 318.

Once the deep neural network 310 has been trained with the training dataset 312, a hacking test 330 can be performed on the trained neural network model 318 to test its robustness. The triangulation testing techniques used in the hacking tests 330 can evolve over time, and any results from the testing can be fed back into the fictionalization strategies and other aspects to improve security. As noted above, for example, an aspect of at least one of: the enumerated categories defined to be fictionalized; the fictionalization process used to fictionalize the enumerated objects; the training framework 314 used to train the model 318; and the hacking test 330 used to test the model 318 can be modified in the systems and processes disclosed herein based on a result from the testing of the trained model 318.

For instance, the hacking test 330 can use triangulation techniques. Such a triangulation test 330 can use known, publicly available facts in the triangulation technique to determine if any target facts can be revealed from within the trained neural network model 318. Because the underlying dataset 311a used to produce the training dataset 312 is known, the triangulation test 330 can readily obtain and use any known, publicly available (i.e., public) facts 311d, which are associated with the underlying patient information in the underlying dataset 311a. The public facts 311d can come from a variety of sources, including news reports, social media platforms, etc.

Using these public facts 311d, the triangulation test 330 can probe the trained neural network model 318 to determine if underlying facts from the underlying dataset 311a can be revealed through the triangulation test 330. This triangulation test 330 can be used to inform further alteration of the underlying dataset 311a with the de-identification and fictionalization steps 311b-c for use in training the neural network.

As noted briefly above, an AI training framework can train a preparatory AI model (57; FIG. 2) to de-identify and/or fictionalize original data to generate synthetic data for a training dataset, which is then used to train a primary AI model. FIG. 7 illustrates a process for training and deploying a preparatory deep neural network 410 for processing an underlying dataset 420 to produce a primary training dataset 422 for use by the primary deep neural network (410; FIG. 6). Features can be similar to those described above with respect to FIG. 6, and the training can be used to train a preparatory AI model (57; FIG. 2) as described above in the computerized system (50) of FIG. 2.

In FIG. 7, the neural network 410 is structured for its task to de-identify and/or fictionalize medical data. Once structured, the neural network is trained using a preparatory dataset 412. In contrast to the previous neural network, the preparatory dataset 412 of medical information has not been processed to remove identifier facts and to fictionalize other facts because this neural network is being trained to perform these de-identification and/or fictionalization steps.

Training of the deep neural network 410 begins by choosing initial weights for the untrained model 416 either by random choice or by pre-training using a deep belief network. The training framework 414 then performs a training cycle, which can then be performed in either a supervised or unsupervised manner.

Supervised learning uses the preparatory dataset 412 to teach models to yield the desired output. The preparatory dataset 412 includes inputs and desired outputs, which allow the model to learn over time, or when the preparatory dataset 412 includes input having known output and the output of the neural network is manually graded.

The training framework 414 of the deep neural network 410 processes the inputs and compares the resulting outputs against a set of expected or desired outputs. Errors are then propagated back through the training framework 414. As training proceeds, the training framework 414 can adjust and change the weights that control the untrained neural network model 416. The training framework 414 can provide tools to monitor how well the untrained model 416 is converging towards a model suitable for generating correct answers based on known input data. The training process repeatedly occurs as the network weights are adjusted to refine the output generated by the neural network. The training process can continue until the neural network reaches a statistically desired accuracy associated with a trained neural network model 418.

Testing 430 as disclosed herein can be used on the trained neural network model 418 to test its robustness. Ultimately, the trained model 418 can then be deployed so the trained model 418 presented with an underlying dataset 420 of medical information can implement the machine learning operations to de-identify and/or fictionalize the medical facts so a primary training dataset 422 can be output for use in training a primary AI model as disclosed herein.

As discussed herein, the goal of the preparatory trained neural network model 418 of FIG. 7 is to prepare a de-identified and fictionalized dataset for machine learning in which the fictionalization steps do not destroy relevant relationships so the primary trained neural network model (318) of FIG. 6 can be accurately trained to produce better results (322) from an end user's input dataset (320). Accordingly, the shared goal for both of the neural network models (318, 418) is to also protect and anonymize the underlying medical information that has been used in the training the neural network models (318, 418).

The foregoing description of preferred and other embodiments is not intended to limit or restrict the scope or applicability of the inventive concepts conceived of by the Applicants. It will be appreciated with the benefit of the present disclosure that features described above in accordance with any configuration or aspect of the disclosed subject matter can be utilized, either alone or in combination, with any other described feature, in any other configuration or aspect of the disclosed subject matter.

In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

1. A method used in a computerized system, the method comprising:
defining, in memory of the computerized system, fact categories including one or more identifier categories and including one or more enumerated categories, the one or more identifier categories categorizing direct identifiers of personally identifiable information, the one or more enumerated categories categorizing enumerated objects being separate from the direct identifiers and being at least related to information that is personally identifiable;
obtaining, with one or more processors of the computerized system, medical facts for a plurality of patients;
vectorizing, with the one or more processors, the medical facts into fact vectors according to the fact categories;
de-identifying, in a de-identification process with the one or more processors, any given one or more of the direct identifiers for the fact vectors in the one or more identifier categories;
fictionalizing, in a fictionalization process with the one or more processors, any given one or more of the enumerated objects for the fact vectors in the one or more enumerated categories;
storing, in the memory, the medical facts resulting from the de-identification process and the fictionalization process as a training dataset;
training, with the one or more processors, an untrained artificial intelligence (AI) model of the computer system into a trained AI model using the training dataset; and
testing, with the one or more processors, the trained AI model for functional utility and resistance to triangulation attacks.

2. The method of claim 1, wherein the one or more identifier categories are selected from the group consisting of patient name, geographical element, a street address, city, county, zip code, date related to health of an individual, date related to identity of an individual, birthdate, date of admission, date of discharge, date of death, exact age of a patient older than 89, telephone number, fax number, e-mail address, social security number, medical record number, health insurance beneficiary number, account number, certificate/license number, vehicle detail, device attribute or serial number, digital identifier, website URL, IP address, biometric element, fingerprint, retinal image, voiceprint, full face photographic image, identifying number, and identifying code.

3. The method of claim 1, wherein the step of de-identifying comprises redacting the any given one or more of the direct identifiers.

4. The method of claim 1, wherein the step of de-identifying t comprises replacing the any given one or more of the direct identifiers as a given direct identifier with an element selected from the group consisting of a de-identifier of the given direct identifier, a code being non-descriptive of the given direct identifier, an abstraction of the given direct identifier, a substitution for the given direct identifier, a truncation of the given direct identifier, a generalization of the given direct identifier, a cryptographic hash generated from the given direct identifier, and an object encrypted from the given direct identifier.

5. The method of claim 1, wherein the one or more enumerated categories are selected from the group consisting of geographic information, temporal information, employment information, education information, socioeconomic information, identity of physician treating a patient, prescription information, and contextual information.

6. The method of claim 1, wherein the step of fictionalizing comprises one or more of:
swapping the any given one or more of the enumerated objects as given enumerated objects in the fact vectors with one another in at least a subset of the medical facts;
shuffling the given enumerated objects in the fact vectors in at least a subset of the medical facts;
randomly reordering the given enumerated objects in the fact vectors in at least a subset of the medical facts;
repeating at least one of the given enumerated objects in the fact vectors throughout at least a subset of the medical facts; and
replacing at least one of the given enumerated objects in at least one of the fact vectors in at least one of the medical facts with at least one of an abstraction of the at least one given enumerated object, a substitution for the at least one given enumerated object, and a generalization of the at least one given enumerated object.

7. The method of claim 1, wherein the step of fictionalizing comprises one or more of:
perturbing the any given one or more of the enumerated objects as given enumerated objects in the fact vectors;
truncating the given enumerated objects in the fact vectors;
truncating a date of the given enumerated objects;
adjusting the given enumerated objects by an increment;
adjusting a numerical value of the given enumerated objects within a range of values;
shifting a date of the given enumerated objects; and
averaging the given enumerated objects in the fact vectors in at least a subset of the medical facts.

8. The method of claim 1, wherein the step of fictionalizing comprises:
clustering the medical facts into clusters based on the fact vectors in two or more of the fact categories; and
fictionalizing the any given one or more of the enumerated objects for the fact vectors in each of the clusters using a same form of the fictionalization process.

9. The method of claim 1, wherein the step of fictionalizing comprises:
identifying quasi-identifiers in the any given one or more of the enumerated objects;
applying K-anonymity to the identified quasi-identifiers.

10. The method of claim 9, wherein the step of applying the K-anonymity to the identified quasi-identifiers comprises reducing distortion of the fact vectors for the any given one or more of the enumerated objects by minimizing a number of the quasi-identifiers identified.

11. The method of claim 9, wherein the step of identifying the quasi-identifiers comprises preserving at least some of the fact vectors for the any given one or more of the enumerated objects by defining generalization hierarchies of the quasi-identifiers.

12. The method of claim 1, wherein the step of fictionalizing comprises preserving a correlation between the fact vectors in the one or more enumerated categories by applying micro-aggregation to the fact vectors.

13. The method of claim 1, wherein the step of fictionalizing comprises:
performing iterative fictionalization of the any given one or more of the enumerated objects for the fact vectors in the one or more enumerated categories;
relaxing constraints used in the fictionalization from an initial iteration to a subsequent iteration; and
balancing anonymity of the training dataset relative to the functional utility of the training dataset at each iteration.

14. The method of claim 13, wherein the step of balancing the anonymity of the training dataset relative to the functional utility of the training dataset at each iteration comprises employing a discernibility metric that measures how many of the medical facts are indistinguishable from each other.

15. The method of claim 1, wherein the step of fictionalizing comprises:
training, with the one or more processors, a preparatory untrained AI model of the computerized system into a preparatory trained AI model; and
fictionalizing the any given one or more of the enumerated objects for the fact vectors at least in the one or more enumerated categories by using the preparatory trained AI model.

16. The method of claim 1, wherein the steps of vectorizing, de-identifying, and fictionalizing comprise using a natural language processing platform of the computer system.

17. The method of claim 1, wherein the step of training the untrained AI model into the trained AI model using the training dataset comprises setting and adjusting weights in repeated processing of the training dataset to converge toward desired outputs by using a neural network training framework of the computer system.

18. The method of claim 1, wherein the step of testing the trained AI model for the functional utility and the resistance to the triangulation attacks comprises testing the trained AI model to reveal any of the medical facts by probing the AI model using public facts associated with the medical facts.

19. The method of claim 1, further comprising modifying, based on a result from the testing of the trained AI model, an aspect of at least one of: the one or more enumerated categories to be fictionalized; the fictionalization process used to fictionalize the enumerated objects; the training of the AI model; and the testing of the AI model.

20. A programmable storage device having program instructions stored thereon for causing a programmable control device to perform a method of claim 1 used in a computerized system.

21. A computerized system, comprising:
a memory storing an untrained artificial intelligence (AI) model and storing fact categories, the fact categories including one or more identifier categories and including one or more enumerated categories, the one or more identifier categories categorizing direct identifiers of personally identifiable information, the one or more enumerated categories categorizing enumerated objects being separate from the direct identifiers and being at least related to information that is personally identifiable;
an interface configured to obtain medical facts for a plurality of patients; and
one or more processors in operable communication with the memory and the interface, the one or more processors being configured to:
vectorize the medical facts into fact vectors according to the fact categories;
de-identify in a de-identification process any given one or more of the direct identifiers for the fact vectors in the one or more identifier categories;
fictionalize in a fictionalization process any given one or more of the enumerated objects for the fact vectors in the one or more enumerated categories;
store the medical facts resulting from the de-identification process and the fictionalization process in the memory as a training dataset;
train, using the training dataset, the untrained AI model into a trained AI model for storage in the memory; and
test the trained AI model for functional utility and resistance to triangulation attacks.
